(19) United States
(12) Patent Application Publication
(10) Pub. No.: US 2023/0293801 A1
(43) Pub. Date: Sep. 21, 2023

(54) Flow Electroporator Device For Therapeutic Targeting Of Circulating Tumor Cells During Hemodialysis

(71) Applicant: ROSEMAN UNIVERSITY OF HEALTH SCIENCES, Las Vegas, NV (US)

(72) Inventors: Thuc T. Le, Las Vegas, NV (US); Yasuyo Urasaki, Las Vegas, NV (US)

(21) Appl. No.: 18/173,698

(22) Filed: Feb. 23, 2023

Related U.S. Application Data
(60) Provisional application No. 63/313,572, filed on Feb. 24, 2022.

(51) Int. Cl. A61M 1/36

(52) U.S. Cl. CPC A61M 1/3687; A61M 1/367; A61M 2202/20; A61M 2205/054

(57) ABSTRACT

The disclosure relates to methods for therapeutically targeting circulating tumor cells during hemodialysis, comprising: connecting a patient's bloodstream to a flow electroporator device, the device comprising: an array of polymeric channels, where the opposing sidewalls of each channel are lined with discontinuous sections of electrodes, interspersed by non-conductive polymer sections to generate sequential electric fields by constant direct current voltages; sequential electric fields with independently regulated intensity, duration, and polarity to induce lysis of circulating tumor cells and deliver chemotherapeutic agents to reduce the viability of circulating tumor cells; and polymeric tubing inlet and outlet for seamless integration into the bloodline tubing of any hemodialysis machine

### RELATED APPLICATIONS

This application claims the benefit of U.S. Provisional Patent Application 63/313,572, filed Feb. 24, 2022, the entire contents of which is incorporated herein by reference in its entirety.

### BACKGROUND

Cancer affects one in two men and one in three women in the United States (US). Approximately 1 out of 25 US citizens is a cancer survivor. The 5-years survival rate for cancer patients is >90% for Stage I, where tumor is localized to a primary site, and <20% for Stage IV, where cancer has spread to secondary sites, or metastasized. Cancer metastasis is the primary cause of cancer-related deaths.

Circulating tumor cells (CTCs) provide the link between primary and secondary tumors. CTCs, or disseminated tumor cells in the bloodstream, are a fundamental prerequisite to cancer metastasis. CTCs can evade the immune system, escape circulation, and develop malignant growths at secondary sites. CTCs have been accepted by the U.S. Food and Drug Administration (FDA) as a prognostic tool for cancer metastasis. Following the initiation of cancer therapy, the number of CTCs ≥5 in a 7.5 ml blood draw predicts poor outcome in terms of progression-free survival and overall survival of patients with various types of cancer.

CTCs exhibit several hallmarks that can be exploited for selective targeting. For example, CTCs have enlarged nuclei due to genetic instability, polyploidy, and high proliferative state. Increased chromosomal DNA content renders CTCs highly susceptible to electroporation-induced nucleus expansion and lysis. In addition, CTCs accumulate excessive cytoplasmic lipid droplets as a defensive mechanism against the lipotoxic condition in the bloodstream. Small-molecule inhibitors of lipid droplet biosynthesis induce autophagic cell death of CTCs. While conventional surgery and radiation therapy are effective at removing primary tumors, secondary tumors are difficult to detect until they become fatal. Thus, there is a need for novel methods of therapeutic targeting of CTCs to prevent cancer metastasis and reduce cancer-related deaths.

### SUMMARY

This disclosure relates to methods to neutralize CTCs during hemodialysis to prevent cancer metastasis and reduce cancer-related deaths. In one aspect, the invention relates to a method that uses a flow electroporator device to induce lysis of CTCs during hemodialysis. The flow electroporator device comprises fluidic channels with embedded electrodes, whose geometric variation determines the electric field intensity and pulse duration. The flow electroporator device can be integrated into the arterial bloodline tubing of a hemodialysis machine and applies localized sequential electrical pulses to neutralize CTCs in extracorporeal blood, thereby avoiding the risk of electrical shock to cancer patients.

In another aspect, the disclosure relates to a method that uses a flow electroporator device to deliver chemotherapeutic agents via electroporation to reduce the viability of CTCs during hemodialysis. The chemotherapeutic agents can be electrolytes, small chemical molecules, proteins, RNAs, or DNAs. Chemotherapeutic agents can be introduced to extracorporeal blood via infusion. The flow electroporator device can deliver chemotherapeutic agents into CTCs using localized sequential electrical pulses. Excess chemotherapeutic agents can be removed by dialysis, thereby minimizing unnecessary exposure to cancer patients. In another aspect, the disclosure relates to a method that uses a flow electroporator device to deliver chemotherapeutic agents via electroporation to reduce the viability of CTCs during hemodialysis. The chemotherapeutic agents can be electrolytes, small chemical molecules, proteins, RNAs, or DNAs. Chemotherapeutic agents can be introduced to extracorporeal blood via infusion. The flow electroporator device can deliver chemotherapeutic agents into CTCs using localized sequential electrical pulses. Excess chemotherapeutic agents can be removed by dialysis, thereby minimizing unnecessary exposure to cancer patients.

In one aspect, the disclosure relates to a method for therapeutically targeting circulating tumor cells during hemodialysis, the method comprising: connecting a patient's bloodstream to a flow electroporator device, the device comprising: (a) an array of polymeric channels, wherein the opposing sidewalls of each channel are lined with discontinuous sections of electrodes, interspersed by non-conductive polymer sections to generate sequential electric fields by constant direct current voltages; (b) sequential electric fields with independently regulated intensity, duration, and polarity to induce lysis of circulating tumor cells and deliver chemotherapeutic agents to reduce the viability of circulating tumor cells; and (c) polymeric tubing inlet and outlet for integration into the bloodline tubing of any hemodialysis machine. The polymer can be acrylonitrile butadiene styrene, polyamide, polycarbonate, polyethylene, polymethyl methacrylate, polypropylene, or polyvinyl chloride. The electrode can be aluminum, chrome, cobalt, copper, gold, magnesium, nickel, palladium, platinum, silver, stainless steel, or titanium. The number of polymeric channels can be between 1 and 1024. The height and width of each channel can be between 1 micrometer and 100 millimeters. The blood flow rate through each channel can be between 0.06 ml/min and 3400 ml/min. The length of the electrodes can be between 1 nanometer and 1 meter. The number of sequential electric fields can be between 1 and 10,000. The electric field intensity can be between 5 mV/cm and 5 kV/cm. The electrical pulse duration can be between 1 picosecond and 10 hours. The circulating tumor cells can be lysed by sequential electrical pulses. The lysis of circulating tumor cells can be enhanced with switched polarity electrical pulses. The lysis of circulating tumor cells can be enhanced with electrolytes. The electrolytes can be sodium, calcium, potassium, chloride, phosphate, or magnesium. The molar concentrations of electrolytes can be between 1 millimolar and 1 molar. The method of claim 1, wherein chemotherapeutic agents are delivered into circulating tumor cells and blood cells using sequential electrical pulses. The chemotherapeutic agents can be electrolytes, small chemical molecules, proteins, RNAs, or DNAs. The lysis of circulating tumor cells and delivery of chemotherapeutic agents can be performed individually, sequentially, simultaneously, or in combination with other methods of purging circulating tumor cells from extracorporeal blood. The debris of lysed circulating tumor cells and excess chemotherapeutic agents can be removed from extracorporeal blood via dialysis.

### BRIEF DESCRIPTION OF THE DRAWINGS

To better understand the present disclosure, it will now be described by way of examples, with reference to the accompanying drawings in which embodiments of the disclosures are illustrated and, together with the description below, explain the principles of the disclosure.

FIGS. 1A-D show nucleus expansion of circulating tumor cells (CTCs) under applied electric fields. FIG. 1A shows a sketch of a microfluidic flow electroporator device that can operate at a maximum flow rate of 0.28 ml/min. FIG. 1B shows expansion of a CTC (arrow) in whole blood under an applied electric field of 400 V/cm. FIG. 1C shows expansion of chromosomal DNA of a CTC under an applied electric field of 400 V/cm. Chromosomal DNA was stained with Hoechst 33342 (blue). FIG. 1D shows nucleus expansion as a function of time of a CTC under a constant electric field of 400 V/cm. CTCs were stained with a DiOC18 lipophilic fluorescent dye, which labeled the cell membrane and cytoplasmic organelles. The nucleus is the compartment without fluorescent staining. FIG. 1E shows quantitative data indicating that nucleus expansion was mainly responsible for CTC expansion under applied electric fields. Error bars are standard deviation of measurement of 10 individual CTCs. FIG. 1F shows the percentage of cell death as a function of applied electric field intensity for CTCs, white blood cells (WBCs), and red blood cells (RBCs). Error bars are standard deviation of triplicate experiments, where approximately 10,000 CTCs, RBCs, WBCs were assayed per experiment. Madison 109 (M109) murine lung carcinoma cells were used as mocked CTCs. Images are taken from Bao et al., Integr. Biol. 2:113-120 (2010), PMID: 20473389.

FIGS. 2A-H are sketches of a flow electroporator device that can operate at flow rates from 0.06 ml to 3400 ml/min. FIG. 2A shows the sketch of a plugged-in flow electroporator device with three localized electric fields, which are physical fields between the opposing negative (black) and positive (red) electrodes. FIG. 2B shows sections of the flow electroporator device. FIG. 2C shows the dimensions in millimeters. FIG. 2D shows the cross-section view of sections G-G and B-B, which are the polyvinyl chloride sections. The internal and external diameters of these sections are 4.8 and 6.4 mm, respectively. FIG. 2E shows the cross-section view of sections E-E, D-D, and C-C, which are the polyvinyl chloride-aluminum composite sections. FIG. 2F shows the cross-section view of section F-F, which is the polymer section. Note the array of four fluidic channels that run continuously through the polymer and polymer-metal composite sections. FIG. 2G shows a side view of the flow electroporator device. FIG. 2H shows the exploded side view of the flow electroporator device to highlight the polymer and metal sections, as well as the array of fluidic channels, of the flow electroporator device.

FIGS. 3A-C are illustrations of an arterial bloodline tubing with an integrated flow electroporator device and its utility for therapeutic targeting of CTCs during hemodialysis. FIG. 3A shows a side view of an arterial bloodline tubing with an integrated flow electroporator device. FIG. 3B shows an exploded side view of an arterial bloodline tubing with an integrated flow electroporator device. The components of the arterial bloodline tubing include: 1a-c: Luer locks; 2a, b: pinch clamps; 3: bloodline tubing; 4: needleless access; 5: arterial pressure pod; 6: arterial pressure pod tubing; 7: pump header tubing; 8: connector; 9: heparin line tubing; 10: flow electroporator device; 11: Luer lock connector. FIG. 3C is an illustration of the hemodialysis process to therapeutically target CTCs using an arterial bloodline tubing with an integrated flow electroporator device. The arterial bloodline allows blood of cancer patients to be removed for cleaning. Therapeutic targeting of CTCs during hemodialysis includes three main steps: (1) infusion of chemotherapeutic agents, (2) electroporation to induce lysis of CTCs and deliver chemotherapeutic agents to reduce the viability of CTCs, and (3) dialysis to remove debris of lysed CTCs and excess chemotherapeutic agents. Gases produced by the electrolysis of extracorporeal blood are removed via an air trap. Cancer-free blood is returned to the patients via a venous bloodline.

FIGS. 4A-H show that a switched polarity transferring pulse reduces the viability of CTCs, but increases the viability of PBMCs. FIG. 4A shows a sketch of a flow electroporator device with three localized electric fields. FIG. 4B shows an example on the electrical pulse durations and field intensities that blood cells experience while traversing the flow electroporator device at a flow rate of 500 ml/min. In this example, the applied voltages were 50V, 40V, and 40V. Blood cells experienced electrical pulse durations of 5 ms (poring pulse), 50 ms (transferring pulse), and 50 ms (transferring pulse) and field intensities of 500 V/cm, 400 V/cm, and 400 V/cm, respectively. FIG. 4C shows another example on the electrical pulse durations and field intensities that blood cells experience while traversing the flow electroporator device at the flow rate of 500 ml/min. In this example, the applied voltages were 50V, 40V, and −40V. Blood cells experienced electrical pulse durations of 5 ms (poring pulse), 50 ms (transferring pulse), and 50 ms (switched polarity transferring pulse) and field intensities of 500 V/cm, 400 V/cm, and −400 V/cm, respectively. FIGS. 4D-I show flow cytometry data on cell viability using propidium iodide (PI) staining. Low PI staining indicates cell death via membrane poration. High PI staining indicates cell death via nucleus poration. FIG. 4D and 4E show the PI staining profile of CTCs after traversing the flow electroporator device under conditions described in FIG. 4B and FIG. 4C, respectively. FIG. 4F compares the PI staining profiles of CTCs treated with conditions described in FIG. 4B (without switched polarity transferring pulse) versus FIG. 4C (with switched polarity transferring pulse). FIG. 4G and 4H show the PI staining profile of peripheral blood mononuclear cells (PBMCs) after traversing the flow electroporator device under conditions described in FIG. 4B and FIG. 4C, respectively. FIG. 4I compares the PI staining profiles of PBMCs treated with conditions described in FIG. 4B (without switched polarity transferring pulse) versus FIG. 4C (with switched polarity transferring pulse). Switched polarity transferring pulse increases CTC death via nucleus poration while decreases PBMC death via membrane poration. HeLa cervical cancer cells are used as mocked CTCs.

FIGS. 5A-I show that electrolytes enhance nucleus poration and lysis of CTCs. FIG. 5A shows the electrical pulse durations and field intensities that blood cells experience while traversing the flow electroporator device at a flow rate of 500 ml/min. Specifically, blood cells experience three electrical pulses: a 5 ms pulse at 300 V/cm, a 50 ms pulse at 200 V/cm, and a 50 ms pulse at −200 V/cm. Applied voltages for the three electric fields were 30V, 20V, and −20V. FIG. 5B shows viability of CTCs following treatment with the flow electroporator device. Cell viability is assayed with crystal violet staining and visualized at low resolution (upper row) and high resolution (lower row). First column shows viability of untreated control HeLa cells in EMEM culturing medium. Second column shows viability of HeLa cells in EMEM culturing medium treated with the flow electroporator device. Third column shows viability of HeLa cells in an electrolyte solution treated with the flow electroporator device. The electrolyte solution is EMEM culturing medium supplemented with NaCl, KCl, and CaCl₂ at final concentrations of 250 mM, 46 mM, and 70 mM, respectively. FIG. 5C shows graphical presentation of HeLa cell viability as a function of treatment conditions. Error bars are standard deviation of triplicate experiments. Single asterisk indicates p-value≤0.01 versus untreated control. Double asterisks indicate p-value≤0.01 versus treatment in EMEM solution. FIG. 5D shows debris of HeLa cells following treatment with the flow electroporator device in an electrolyte solution. The membranes of HeLa cells were stained with Annexin V conjugate (green) and the chromosomal DNA were stained with propidium iodide (red). Images are taken with dual-color confocal fluorescence microscopy. FIG. 5E shows Western blots of released housekeeping proteins HSP60 and β-actin for untreated control HeLa cells (first lane), HeLa cells treated with Bicine/CHAPS lysis buffer (second lane), and HeLa cells in an electrolyte solution treated with the flow electroporator device (third lane). FIGS. 5F-I show flow cytometry data on cell viability using propidium iodide staining. FIG. 5F shows the PI staining profiles of untreated control HeLa cells (black line) versus HeLa cells treated at 95° C. for 3 minutes (red line). FIG. 5G shows the PI staining profiles of untreated control HeLa cells (black line) versus HeLa cells in an electrolyte solution treated with the flow electroporator device (red line). FIG. 5H shows the PI staining profiles of untreated control HeLa cells (black line) versus peripheral blood mononuclear cells (PBMCs) treated at 95° C. for 3 minutes (red line). FIG. 5I shows the PI staining profiles of untreated control PBMCs (black line) versus PBMCs in an electrolyte solution treated with the flow electroporator device (red line).

FIGS. 6A-G show drug delivery via electroporation. FIGS. 6A-C show doxorubicin induces cell cycle arrest of HeLa cells. Cell cycle analysis is performed using flow cytometry and propidium iodide staining of chromosomal DNA. FIG. 6A shows flow cytometry cell cycle analysis of control untreated HeLa cells. FIG. 6B shows flow cytometry cell cycle analysis of HeLa cells treated with 40 nM doxorubicin for 24 hrs. FIG. 6C shows cell cycle distribution as a function of doxorubicin treatment. Error bars are standard deviation of triplicate experiments. Asterisk indicates p-value≤0.01 versus untreated control. FIGS. 6D-G show increased delivery of doxorubicin into HeLa cells via electroporation. FIG. 6D shows flow cytometry analysis of doxorubicin fluorescence (FL2-A) of untreated control HeLa cells. FIG. 6E shows flow cytometry analysis of doxorubicin fluorescence of HeLa cells in EMEM culturing medium incubated for 30 minutes with 40 nM doxorubicin. FIG. 6F shows flow cytometry analysis of doxorubicin fluorescence of HeLa cells in EMEM culturing medium incubated with 40 nM doxorubicin and treated with the flow electroporator device at a flow rate of 500 ml/min and sequential electric fields of 300 V/cm, 200 V/cm, −200 V/cm. FIG. 6G shows graphical presentation of doxorubicin uptake as a function of electroporation (EP). Data are normalized to 1 for doxorubicin fluorescence of HeLa cells incubated with 40 nM doxorubicin without electroporation. Error bars are standard deviation of triplicate experiments. Asterisk indicates p-value≤0.01 versus no electroporation.

FIGS. 7A-H show A922500, a DGAT1 inhibitor, reduces the viability of CTCs in blood plasma. FIG. 7A shows viability of HeLa cells following incubation with blood plasma for 24 hrs. Cell viability is assayed with crystal violet staining and visualized at low resolution (left column) and high resolution (right column). First row shows viability of HeLa cells cultured in EMEM medium for 24 hrs. Second row shows viability of HeLa cells incubated with blood plasma for 24 hrs. FIG. 7B shows cell viability as a function of culturing conditions. Error bars are standard deviation of triplicate experiments. Asterisk indicates p-value≤0.01 versus culturing in EMEM medium. FIG. 7C shows capillary isoelectric focusing immunoblots of autophagosome biomarker proteins LC3A and LC3B in HeLa cells cultured in EMEM (left lane) or blood plasma (right lane). FIG. 7D shows confocal fluorescent images of HeLa cells stained with Hoechst 33342, which labels chromosomal DNA, and a fluorescent dye (green), which labels autophagosomes. Control HeLa cells cultured in EMEM medium is presented in the top panel. HeLa cells incubated with blood plasma for 24 hrs is presented in the bottom panel. FIG. 7E shows confocal fluorescent images of HeLa cells stained blue for chromosomal DNA, green for actin, and red for intracellular lipid droplets. Images are control untreated HeLa cells cultured with EMEM medium (first panel); HeLa cells cultured in EMEM medium and treated with 10 μM A922500, a small-molecule inhibitor of diacylglycerol O-acyltransferase 1 (DGAT1), for 24 hrs (second panel); HeLa cells incubated with blood plasma for 24 hrs (third panel); and HeLa cells incubated with blood plasma and 10 μM A922500 for 24 hrs (fourth panel). FIGS. 7F-H show viability of HeLa cells following incubation with blood plasma or blood plasma and A922500. Cell viability is assayed with crystal violet staining and visualized at low resolution (FIG. 7F) and high resolution (FIG. 7G). FIG. 7H shows graphical presentation of HeLa cell viability as a function of treatment conditions. Error bars are standard deviation of triplicate experiments. Single asterisk indicates p-value≤0.01 versus untreated control HeLa cells cultured with EMEM. Double asterisks indicate p-value≤0.01 versus HeLa cells incubated with blood plasma for 24 hrs.

FIGS. 8A-D shows gene delivery via electroporation. FIG. 8A shows flow cytometry assay of GFP fluorescence (FL1-A) for HeLa cells incubated with a pCMV-GFP plasmid in EMEM medium without electroporation. FIG. 8B shows flow cytometry assays of GFP fluorescence for HeLa cells incubated with a pCMV-GFP plasmid in EMEM medium with electroporation. Electroporation is performed using the flow electroporator device at a flow rate of 500 ml/min and sequential electric field intensities of 300V/cm, 200 V/cm, and −200 V/cm. FIGS. 8C and 8D show confocal fluorescence images of chromosomal DNA (blue) and GFP (green) of HeLa incubated with a pCMV-GFP plasmid in EMEM medium without electroporation (FIG. 8C) or HeLa cells incubated with a pCMV-GFP plasmid in EMEM medium with electroporation (FIG. 8D).

### DETAILED DESCRIPTION

The present disclosure is directed toward a flow electroporator device that therapeutically targets circulating tumor cells (CTCs) during hemodialysis. In particular, the flow electroporator device uses sequential electrical pulses to induce lysis of CTCs and deliver chemotherapeutic agents to reduce the viability of CTCs.

Therapeutic targeting of CTCs during hemodialysis provides a safe and effective means to neutralize them in extracorporeal blood of cancer patients. For therapeutic targeting of CTCs during hemodialysis, localized electric fields are applied to extracorporeal blood to induce lysis of CTCs, thereby avoiding the risk of electrical shock to cancer patients. In addition, chemotherapeutic agents are delivered via electroporation in extracorporeal blood to reduce the viability of CTCs. Excess chemotherapeutic agents are removed via dialysis, thereby minimizing the risk of unnecessary exposure to cancer patients. Therapeutic technologies that exploit hallmarks of CTCs to neutralize them before they have a chance to form secondary tumors could prevent cancer metastasis and reduce cancer-related deaths.

Hemodialysis is an FDA-approved procedure that uses a dialyzer as an artificial kidney to clean the blood of patients who suffer from end-stage renal disease. In 2018, approximately 518,749 patients received hemodialysis in the US. A vascular access allows blood to travel through soft tubes to the dialysis machine where it is cleaned as it passes through a dialyzer.

The present disclosure relates generally to cancer therapy, and more specifically to methods for therapeutic targeting of CTCs during hemodialysis. More particularly, the present invention is directed to a flow electroporator device that reduces the viability of CTCs during hemodialysis. Elimination of CTCs from the bloodstream can reduce cancer metastasis and increase survivability of cancer patients. Another advantage of the methods described herein is the reduction of side effects for patients undergoing chemotherapy treatment.

In one aspect, the disclosure is directed to a flow electroporator device that neutralizes CTCs during hemodialysis, while leaving the viability of blood cells unaffected. The flow electroporator device can comprise fluidic channels with embedded electrodes, whose geometric variation can determine the electric field intensity and pulse duration. The flow electroporator device can be integrated into the arterial bloodline tubing of a hemodialysis machine. In one aspect, the disclosure relates to a two-pronged approach to CTC elimination that includes: (1) application of localized electrical pulses to induce lysis of CTCs and (2) delivery of chemotherapeutic agents via electroporation to reduce the viability of CTCs. Such therapeutic procedures can be conducted in the extracorporeal blood of cancer patients either simultaneously, sequentially, individually or in combination. Excess chemotherapeutic agents and CTC debris can be removed via dialysis. Consequently, side effects are expected to be minimal because cancer patients are not exposed to localized electric fields or chemotherapeutic agents.

Diseases or other medical conditions for which the embodiments described herein are applicable include, but are not limited to, any of a variety of cancers or other neoplastic conditions, including, for example, epithelial cell cancers such as lung, ovarian, cervical, endometrial, breast, brain, colon and prostate cancers. Also included are gastrointestinal cancer, head and neck cancer, non-small cell lung cancer, cancer of the nervous system, kidney cancer, genital-urinary cancer, bladder cancer, melanoma and leukemia. In addition, the embodiments described herein are equally applicable to treatment of non-malignant tumors in an individual (e.g., neurofibromas, meningiomas, and schwannomas).

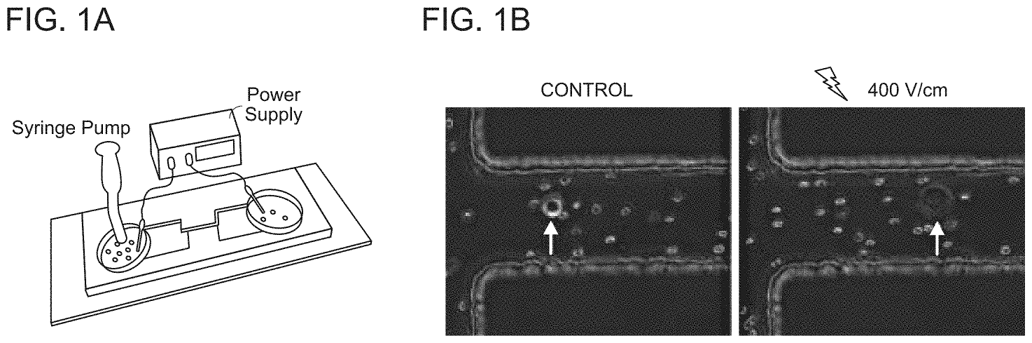

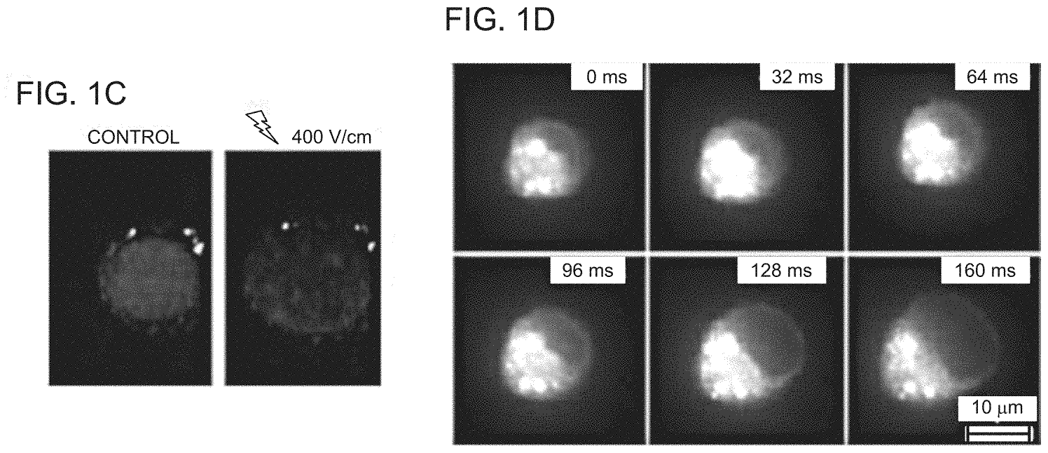

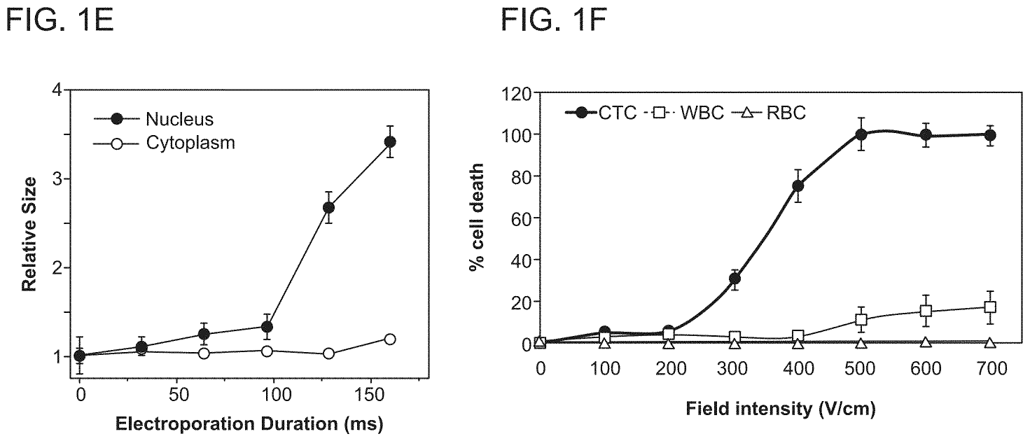

The embodiments described herein exploit the differences between CTCs, red blood cells (RBCs) and white blood cells (WBCs) to selectively neutralize CTCs, while leaving RBCs and WBCs unaffected. On the one hand, CTCs have enlarged nuclei due to genetic instability, polyploidy, and high proliferative state. Increased chromosomal DNA content renders CTCs highly susceptible to electroporation-induced nucleus expansion, nucleus poration, and lysis (FIGS. 1A-E). In contrast, RBCs lack a cell nucleus and have only the plasma membrane as a structural component. WBCs are nucleated and derived from hematopoietic stem cells in the bone marrow. WBCs are non-proliferative under normal condition and have small and tightly packed nuclei. Electrical pulses are known to induce membrane poration in most cell types. However only CTCs, but not WBCs nor RBCs, exhibit nucleus poration under applied electric fields. Nucleus poration under applied electric fields is a mechanism that selectively induces lysis of CTCs while having no observable effect on the viability of RBCs or WBCs (FIG. 1F).

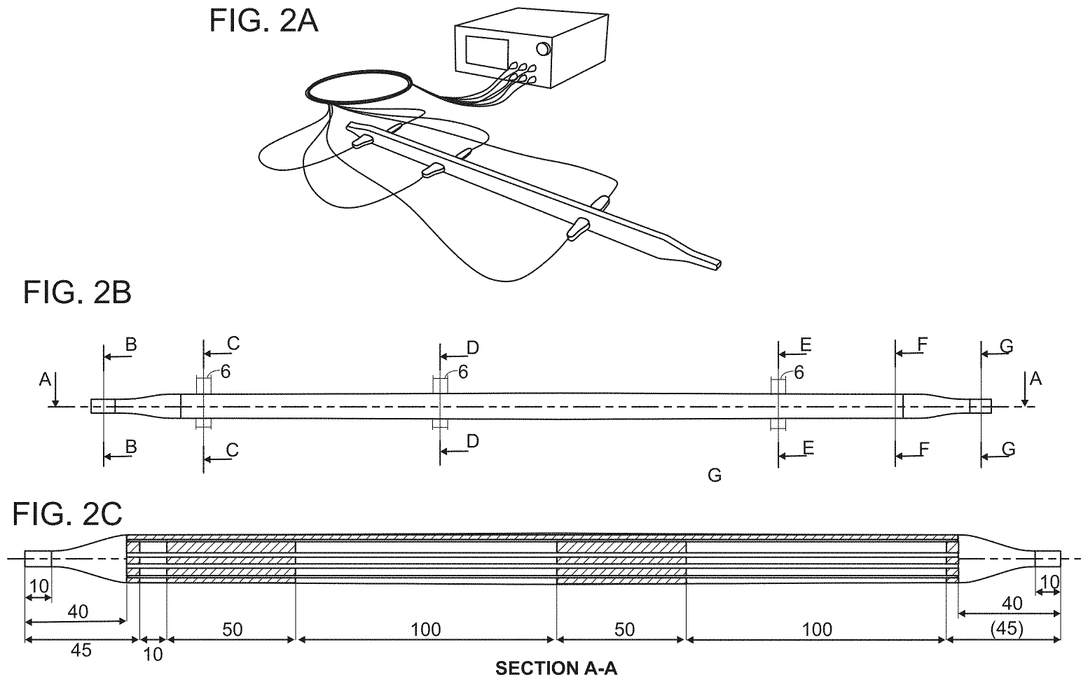

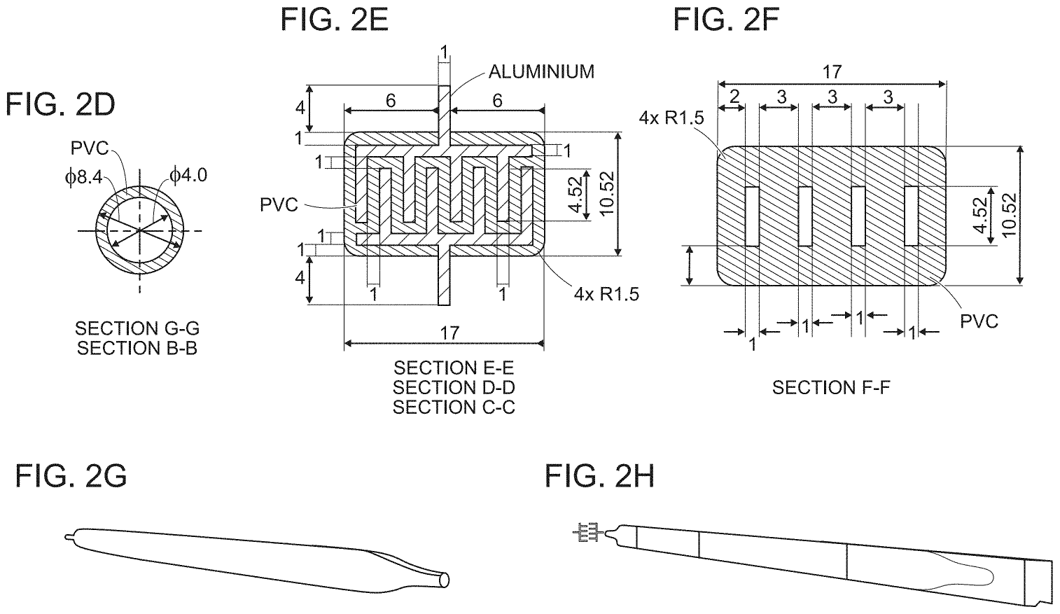

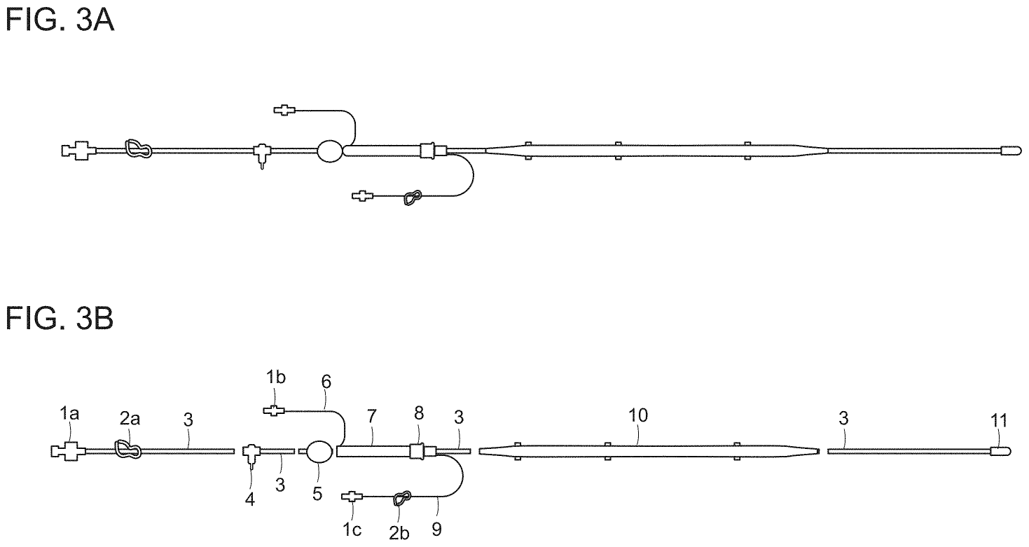

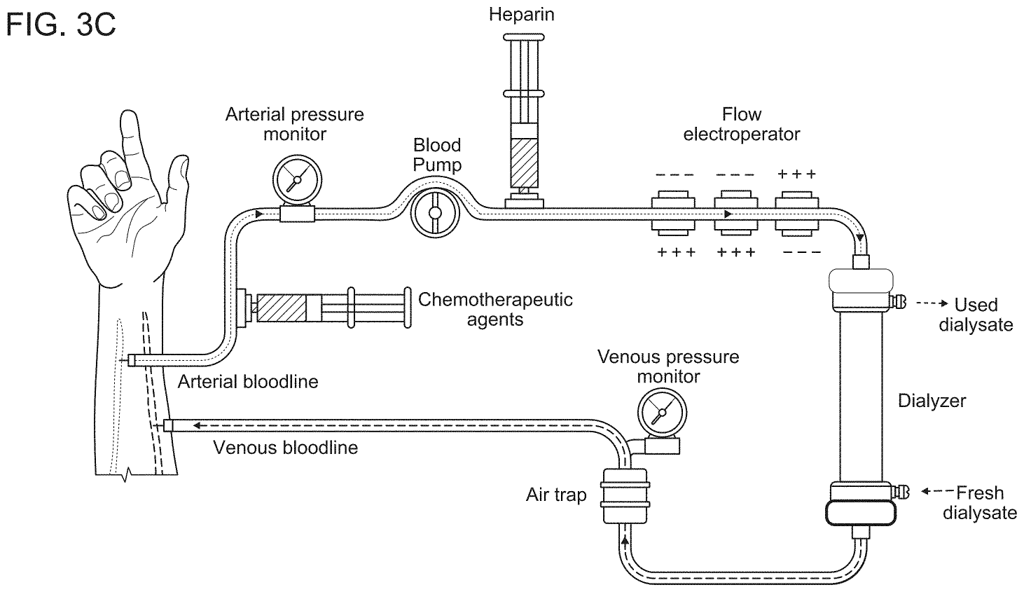

In one embodiment, a flow electroporator device selectively induces lysis to CTCs while leaving RBCs and WBCs unharmed. The flow electroporator device can be made of polymer-metal composites and comprise an array of four fluidic channels with embedded electrodes (FIGS. 2A-H). All fluidic channels can have the height and width dimensions of 4.52 mm and 1 mm, respectively (FIGS. 2E & 2F). The electrodes in each fluidic channel can be separated by 1 mm and cover the entire height of the sidewalls of 4.52 mm (FIG. 2E). The flow electroporator device can provide three sequential electric fields, which are delineated by the lengths of the embedded electrodes of 10 mm, 100 mm, and 100 mm (FIGS. 2A, 2B, 2C, 2G & 2H). The aluminum overhangs can be used to mount the clip-on electrodes. The three sequential electric fields can be separated by two non-conductive PVC sections of 50 mm per section (FIG. 2C). Furthermore, the entrance and exit of the flow electroporator device are made of PVC tubing with internal and external diameters of 4.8 mm and 6.4 mm, respectively, which permit seamless integration of the flow electroporator device into the arterial bloodline tubing of any hemodialysis machine (FIGS. 3A-C).

The geometric variation of the fluidic channels determines the electric field intensity and pulse duration. The rectangular shape of the fluidic channels ensures that all blood cells and CTCs that traverse the flow electroporator device experience uniform electric field intensities and same pulse durations. The constant electric field intensity within each fluidic channel is calculated using the equation:

$$E = \frac{V}{d}$$

where EE is the electric field intensity; VV is the applied voltage; and dd is the distance between the positive and negative electrodes.

The pulse duration that each blood cell experience while traversing each electric field is calculated using the equation:

$$t = \frac{H \times W \times L}{Q}$$

where tt is the pulse duration; HH is the height of the fluidic channel; WW is the width of the fluidic channel; LL is the length of the embedded electrode; and QQ is the volumetric flow rate.

For applied sequential voltages of 50V, 40V, and 40V and a volumetric flow rate of 500 ml/min, blood cells and CTCs that traverse this flow electroporator device experience sequential electric field intensities of 500 V/cm, 400 V/cm, and 400 V/cm and pulse durations of approximately 5 ms, 50 ms, and 50 ms, respectively.

The sequential electric fields with varying field intensities and pulse durations of the flow electroporator device are purposefully designed. First, blood cells and CTCs experience a high electric field intensity for a short duration. This first pulse is known as the “poring pulse”, which opens pores on cell membrane. Second, blood cells and CTCs experience a low electric field intensity for a long duration. This second pulse is known as the “transferring pulse”, which permits transferring of charged materials across cell and nuclear membranes. Third, blood cells and CTCs might experience another “transferring pulse” with the same electrical polarity as the first and second pulses. Alternately, blood cells and CTCs might experience a “switched polarity transferring pulse”. Polarity at any electric field within the flow electroporator device might be switched by reversing the negative and positive clip-on electrodes. The “switched polarity transferring pulse” is generally used to seal pores on cell membrane to improve cell viability. The three independent electric field intensities and pulse durations provide opportunities to explore conditions that maximize the lysis of CTCs while minimizing any effect on the viability of blood cells.

In another aspect, the disclosure relates to a flow electroporator device for drug and gene delivery. In one embodiment, the flow electroporator device is used to deliver doxorubicin to CTCs and blood cells. Doxorubicin is a chemotherapeutic agent in a class of antitumor antibiotics that are extracted from Streptomyces bacterium. It inhibits the replication of chromosomal DNA and growth of cancer cells by blocking an enzyme called topoisomerase 2. Doxorubicin is approved for medical use by the US FDA since 1974. Doxorubicin is used to treat cancers of the bladder, breast, stomach, lung, ovaries, thyroid, soft tissue sarcoma, multiple myeloma, as well as some leukemias and Hodgkin's lymphoma. Doxorubicin is commonly administered by continuous intravenous infusion. The flow electroporator device is expected to deliver doxorubicin to both CTCs and blood cells via electroporation. However, CTCs are highly susceptible to doxorubicin-induced cell death due to their high proliferative state. In contrast, doxorubicin minimally affects the viability of blood cells due to their non-proliferative state under normal condition.

In another embodiment, the flow electroporator device is used to deliver A922500, a small molecular inhibitor of diacylglycerol O-acyltransferase 1 (DGAT1), to CTCs and blood cells in extracorporeal blood during hemodialysis. The bloodstream is a highly toxic environment to CTCs of epithelial origins due to the abundance of bioactive lipids and free fatty acids. As a defensive mechanism, CTCs convert bioactive lipids and free fatty acids into neutral triacylglycerol and store them in cytoplasmic lipid droplets. DGAT1 is an enzyme that catalyzes the conversion of diacylglycerol and fatty acyl-CoA to triacylglycerol. The flow electroporator device is expected to deliver A922500 to both CTCs and blood cells via electroporation. Inhibition of DGAT1 with A922500 suppresses the biosynthesis of triacylglycerol and cytoplasmic lipid droplet accumulation and reduces the viability of CTCs in blood plasma. In contrast, blood cells do not have the capability to store cytoplasmic lipid droplets and are generally unaffected by A922500.

In another embodiment, the flow electroporator device is used for the transfection of CTCs and blood cells in extracorporeal blood during hemodialysis. Transfection is a process of introducing nucleic acids into eukaryotic cells. Several nucleic acid-based therapeutics have emerged for cancer treatment in recent years. RNA interference (RNAi) using small interfering RNA molecules (siRNAs) has been shown to be highly effective in silencing cancer promoting genes and inhibiting tumor growth. Alternatively, anticancer genes, when overexpressed ectopically, have been shown to specifically destroy tumor cells without harming normal cells. For example, TNF-related apoptosis-inducing ligand (TRAIL), a cytokine produced by PBMCs, induces apoptosis primarily in tumor cells. TRAIL-based gene delivery to PBMCs is a viable approach for therapeutic targeting of CTCs, as well as solid tumors. Furthermore, mRNA vaccines hold tremendous promises for cancer immunotherapy. An mRNA vaccine uses strands of mRNA to instruct cells to produce proteins on the surface of a cancer cell. Once the immune system learns to recognize the proteins, it can create antibodies or T cells that fight and destroy the cancer cells. The flow electroporator device uses electroporation to deliver nucleic acid-based therapeutics to CTCs and blood cells during hemodialysis.

The methods disclosed herein utilize a flow electroporator device to induce lysis to CTCs and deliver chemotherapeutic agents to reduce the viability of CTCs. The flow electroporator device can be made of polymer-metal composites that comprise any medical-grade polymers and metals. Medical-grade polymers include, but are not limited to, acrylonitrile butadiene styrene, polyamide, polycarbonate, polyethylene, polymethyl methacrylate, polypropylene, and polyvinyl chloride. Medical-grade metals include, but are not limited to, aluminum, chrome, cobalt, copper, gold, magnesium, nickel, palladium, platinum, silver, stainless steel, and titanium.

The flow electroporator device can comprise between about 1 to 1024 channels with embedded electrodes. For example, the flow electroporator device can comprise at least about 1, 5, 10, 15, 20, 25, 30, 35, 40, 45, 50, 55, 60, 65, 70, 75, 80, 85, 90, 95, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, or 1020 channels with embedded electrodes. The flow electroporator device can comprise between about 1 to 100, 1 to 200, 1 to 300, 1 to 400, 1 to 500, 1 to 600, 1 to 700, 1 to 800, 1 to 900, 1 to 1000, or 1 to 1100 channels.

The height and width of each channel can be from about 1 μm to 100 mm (e.g., from about 1 μm to 1, about 1 μm to 10 mm, about 1 μm to 20 mm, about 1 μm to 30 mm, about 1 μm to 40 mm, about 1 μm to 50 mm, about 1 μm to 60 mm, about 1 μm to 70 mm, about 1 μm to 80 mm, about 1 μm to 90 mm, about 1 μm to 100 mm, or about 1 μm to 110 mm). For example, the height and width of each channel can be at least about 1 μm, 50 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1 mm, 10 mm, 20 mm, 30 mm, 40 mm, 50 mm, 60 mm, 70 mm, 80 mm, 90 mm, or 100 mm.

The lengths of the embedded electrodes can be from about 1 nm to 1 m (e.g., about 10 nm to 1 m, about 20 nm to about 1 m, about 50 nm to about 1 m, about 100 nm to about 1 m, about 200 nm to about 1 m, about 400 nm to about 1 m, about 600 nm to about 1 m, about 800 nm to about 1 m, about 1 um to about 1 m, about 50 um to about 1 m, about 100 um to about 1 m, about 200 um to about 1 m, about 400 um to about 1 m, about 600 um to about 1 m, about 800 um to about 1 m, about 1 mm to about 1 m, about 50 mm to about 1 m, about 100 mm to about 1 m, about 200 mm to about 1 m, about 400 mm to about 1 m, about 600 mm to about 1 m, about 800 mm to about 1, about 1 cm to about 1 m, about 50 cm to about 1 m, about 100 cm to about 1 m, about 200 cm to about 1 m, about 400 cm to about 1 m, about 600 cm to about 1 m, about 800 cm to about 1 m, about 1 nm to about 1 um, about 1 um to about 1 mm, about 1 mm to about 1 cm, or about 1 cm to about 1 m). For example, the lengths of the embedded electrodes can be at least about 1 nm, 100 nm, 200 nm, 300 nm, 400 nm, 500 nm, 600 nm, 700 nm, 800 nm, 900 nm, 1 um, 100 um, 200 um, 300 um, 400 um, 500 um, 600 um, 700 um, 800 um, 900 um, 1 mm, 100 mm, 200 mm, 300 mm, 400 mm, 500 mm, 600 mm, 700 mm, 800 mm, 900 mm, 1 cm, 100 cm, 200 cm, 300 cm, 400 cm, 500 cm, 600 cm, 700 cm, 800 cm, 900 cm, or 1 m.

The number of electric fields can be from about 1 to about 10,000 (e.g., about 1 to about 100, about 1 to about 500, about 1 to about 1000, about 1 to about 2000, about 1 to about 3000, about 1 to about 4000, about 1 to about 5000, about 1 to about 6000, about 1 to about 7000, about 1 to about 8000, about 1 to about 9000, about 1 to about 10,000). For example, the number of electric fields can be at least about 1, 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, 9000, 9200, 9400, 9600, 9800, or 10,000.

The electric fields can be separated by non-conductive polymer sections of between about 1 nm to about 1 m (e.g., between about 10 nm to about 1 m, 100 nm to about 1 m, 200 nm to about 1 m, 400 nm to about 1 m, 600 nm to about 1 m, 800 nm to about 1 m, 1 um to about 1 m, 10 um to about 1 m, 100 um to about 1 m, 200 um to about 1 m, 400 um to about 1 m, 600 um to about 1 m, 800 um to about 1 m, 1 mm to about 1 m, 10 mm to about 1 m, 100 mm to about 1 m, 200 mm to about 1 m, 400 mm to about 1 m, 600 mm to about 1 m, 800 mm to about 1 m, 1 cm to about 1 m, 10 cm to about 1 m, 100 cm to about 1 m, 200 cm to about 1 m, 400 cm to about 1 m, 600 cm to about 1 m, 800 cm to about 1 m, 1 cm to about 1 m, 1 um to about 1 m, 1 mm to about 1 m, 1 cm to about 1 m, 1 nm to about 1 um, 1 um to about 1 mm, 1 mm to about 1 cm, 1 cm to about 1 m). For example, the electric fields can be separated by non-conductive polymer sections of at least about 1 nm, 100 nm, 200 nm, 300 nm, 400 nm, 500 nm, 600 nm, 700nm, 800 nm, 900 nm, 1 um, 100 um, 200 um, 300 um, 400 um, 500 um, 600 um, 700 um, 800 um, 900 um, 1 mm, 100 mm, 200 mm, 300 mm, 400 mm, 500 mm, 600 mm, 700 mm, 800 mm, 900 mm, 1 cm, 100 cm, 200 cm, 300 cm, 400 cm, 500 cm, 600 cm, 700 cm, 800 cm, 900 cm, or 1 m.

The electric field intensity can be from about 5 mV/cm to about 5000 V/cm (e.g., about 5 mV/cm to about 5000 V/cm, 10 mV/cm to about 5000 V/cm, 20 mV/cm to about 5000 V/cm, 40 mV/cm to about 5000 V/cm, 60 mV/cm to about 5000 V/cm, 80 mV/cm to about 5000 V/cm, 100 mV/cm to about 5000 V/cm, 200 mV/cm to about 5000 V/cm, 400 mV/cm to about 5000 V/cm, 600 mV/cm to about 5000 V/cm, 800 mV/cm to about 5000 V/cm, 1 V/cm to about 5000 V/cm, 100 V/cm to about 5000 V/cm, 200 V/cm to about 5000 V/cm, 500 V/cm to about 5000 V/cm, 1000 V/cm to about 5000 V/cm, 1500 V/cm to about 5000 V/cm, 2000 V/cm to about 5000 V/cm, 2500 V/cm to about 5000 V/cm, 3000 V/cm to about 5000 V/cm, 3500 V/cm to about 5000 V/cm, 4000 V/cm to about 5000 V/cm, 4500 V/cm to about 5000 V/cm). For example, the electric field intensity can be at least about 5 mV/cm, 10 mV/cm, 20 mV/cm, 30 mV/cm, 40 mV/cm, 50 mV/cm, 60 mV/cm, 70 mV/cm, 80 mV/cm, 90 mV/cm, 100 mV/cm, 150 mV/cm, 200 mV/cm, 250 mV/cm, 300 mV/cm, 350 mV/cm, 400 mV/cm, 450 mV/cm, 500 mV/cm, 550 mV/cm, 600 mV/cm, 650 mV/cm, 700 mV/cm, 750 mV/cm, 800 mV/cm, 850 mV/cm, 900 mV/cm, 950 mV/cm, 1 V/cm, 100 V/cm, 200 V/cm, 300 V/cm, 400 V/cm, 500 V/cm, 600 V/cm, 700 V/cm, 800 V/cm, 900 V/cm, 1000 V/cm, 1100 V/cm, 1200 V/cm, 1300 V/cm, 1400 V/cm, 1500 V/cm, 1600 V/cm, 1700 V/cm, 1800 V/cm, 1900 V/cm, 2000 V/cm, 2100 V/cm, 2200 V/cm, 2300 V/cm, 2400 V/cm, 2500 V/cm, 2600 V/cm, 2700 V/cm, 2800 V/cm, 2900 V/cm, 3000 V/cm, 3100 V/cm, 3200 V/cm, 3300 V/cm, 3400 V/cm, 3500 V/cm, 3600 V/cm, 3700 V/cm, 3800 V/cm, 3900 V/cm, 4000 V/cm, 4100 V/cm, 4200 V/cm, 4300 V/cm, 4400 V/cm, 4500 V/cm, 4600 V/cm, 4700 V/cm, 4800 V/cm, 4900 V/cm, or 5000 V/cm.

The pulse duration can be from about 1 picosecond to 10 hours (e.g., about 100 picosecond to about 10 hours, about 500 picosecond to about 10 hours, about 1 nanosecond to about 10 hours, about 500 nanoseconds to about 10 hours, about 1 microsecond to about 10 hours, about 500 microseconds to about 10 hours, about 1 millisecond to about 10 hours, about 500 microseconds to about 10 hours, about 1 millisecond to about 10 hours, about 500 milliseconds to about 10 hours, about 1 second to about 10 hours, about 30 seconds to about 10 hours, about 1 minute to about 10 hours, about 1 hour to about 10 hours, about 2 hours to about 10 hours, about 3 hours to about 10 hours, about 4 hours to about 10 hours, about 5 hours to about 10 hours, about 6 hours to about 10 hours, about 7 hours to about 10 hours, about 8 hours to about 10 hours, about 9 hours to about 10 hours). For example, the pulse duration can be at least about 1 picosecond, 100 picosecond, 200 picoseconds, 400 picoseconds, 600 picoseconds, 800 picoseconds, 1 nanosecond, 100 nanoseconds, 200 nanoseconds, 400 nanoseconds, 600 nanoseconds, 800 nanoseconds, 1 microsecond, 100 microseconds, 200 microseconds, 400 microseconds, 600 microseconds, 800 microseconds, 1 second, 10 seconds, 20 seconds, 40 seconds, 60 seconds, 10 minutes, 20 minutes, 40 minutes, 60 minutes, 2 hours, 3 hours, 4 hours, 5 hours, 6 hours, 7 hours, 8 hours, 9 hours, or 10 hours.

The volumetric flow rate can be from about 0.1 ml/min to 3.5 L/min (e.g., about 0.5 ml/min to 3.5 L/min, 1.0 ml/min to 3.5 L/min, 10 ml/min to 3.5 L/min, 50 ml/min to 3.5 L/min, 100 ml/min to 3.5 L/min, 200 ml/min to 3.5 L/min, 400 ml/min to 3.5 L/min, 600 ml/min to 3.5 L/min, 800 ml/min to 3.5 L/min, 1 L/min to 3.5 L/min, 1.5 L/min to 3.5 L/min, 2 L/min to 3.5 L/min, 2.5 L/min to 3.5 L/min, or 3 L/min to 3.5 L/min). For example, the volumetric flow rate can be at least about 0.1 ml/min, 0.5 ml/min, 1 ml/min, 10 ml/min, 15 ml/min, 20 ml/min, 25 ml/min, 30 ml/min, 35 ml/min, 40 ml/min, 45 ml/min, 50 ml/min, 55 ml/min, 60 ml/min, 65 ml/min, 70 ml/min, 75 ml/min, 80 ml/min, 85 ml/min, 90 ml/min, 95 ml/min, 100 ml/min, 200 ml/min, 300 ml/min, 400 ml/min, 500 ml/min, 600 ml/min, 700 ml/min, 800 ml/min, 900 ml/min, 1 L/min, 1.2 L/min, 1.4 L/min, 1.6 L/min, 1.8 L/min, 2.0 L/min, 2.2 L/min, 2.4 L/min, 2.6 L/min, 2.8 L/min, 3.0 L/min, 3.1 L/min, 3.2 L/min, 3.3 L/min, 3.4 L/min, or 3.5 L/min.

The number of electric fields with switched polarity can be from about 1 to about 10,000 (e.g., about 1 to about 500, about 1 to about 1000, about 1 to about 1500, about 1 to about 2000, about 1 to about 2500, about 1 to about 3000, about 1 to about 4000, about 1 to about 4500, about 1 to about 5000, about 1 to about 5500, about 1 to about 6000, about 1 to about 6500, about 1 to about 7000, about 1 to about 7500, about 1 to about 8000, about 1 to about 8500, about 1 to about 9000, about 1 to about 9500, about 1 to about 10,000, about 1000 to about 10,000, about 2000 to about 10,000, about 3000 to about 10,000, about 4000 to about 10,000, about 5000 to about 10,000, about 6000 to about 10,000, about 7000 to about 10,000, about 8000 to about 10,000, or about 9000 to about 10,000). For example, the number of electric fields with switched polarity can be about 1, 50, 100, 150, 200, 250, 300, 350, 400, 450, 500, 550, 600, 650, 700, 750, 800, 850, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10,000.

Electrical signals play an important role in the regulation of cell division, migration, and differentiation. Electrical impulse and potential are associated with critical physiological processes such as muscle contraction, neurotransmission, hearing, and organogenesis. Application of external electrical signals has been shown to promote contraction of cardiac muscle, regeneration of damage spinal nerves, neuronal migration, and wound healing. In addition, deep brain stimulation is a therapy that uses electrical stimulation to treat Parkinson's disease. Electrical signals, together with biochemical and molecular signals, control vital aspects of biological systems in health and disease. Electrochemotherapy is a therapeutic approach to cancer treatment that combines anticancer agents with high electrical voltage to target solid tumors. Electroporation via application of electrical fields alone is also successful at disrupting cancer cell replication in animal xenograft models with a wide range of human tumors. Application of electric fields by themselves or together with chemotherapeutic agents can be an effective means for the treatment of solid tumors. The present disclosure provides application of electric fields to induce lysis of CTCs or deliver chemotherapeutic agents to reduce the viability of CTCs.

Hemodialysis is a procedure to remove waste products from extracorporeal blood when the kidneys are failing to perform their physiological function. In hemodialysis, counter current flows between dialysate and extracorporeal blood are separated by a semi-permeable membrane. Waste products of metabolism in the blood move across the semi-permeable membrane into dialysate from high concentration to low concentration. Hemodialysis presents an opportunity to therapeutically target CTCs with no exposure of human body to electrical shock or chemotherapeutic agents. As outlined in FIG. 3C, chemotherapeutic agents and localized electric fields can be introduced to extracorporeal blood to target CTCs. Excess chemotherapeutic agents and CTC debris are removed from blood via dialysis. Air bubbles that arise from electrolysis are removed by an air trap. Patients are not exposed to the risks of electrical shock, air embolism, or excess chemotherapeutic agents.

### EXAMPLES

### Example 1: Selective Purging of CTCs Using Nucleus Poration

Nucleus enlargement and increased plasticity of the nuclear membrane are hallmarks of cancer cells during malignant transformation. Nucleus enlargement is associated with increased chromosomal DNA content due to genetic instability, polyploidy, and high proliferative state of cancer cells. Increased plasticity of the nuclear membrane is associated with cancer cell motility and invasion. Changes in the nuclear content and mechanics render malignant cancer cells highly susceptible to electroporation-induced nucleus expansion and lysis.

Nucleus expansion is a reliable biomarker to identify CTCs in blood draws. Under an applied electric field of 400 V/cm, the nuclei of CTCs expand more than three times compared to those of untreated control (FIGS. 1A-E). In contrast, RBCs and WBCs do not exhibit any observable nucleus expansion under the same electric field (FIG. 1B). RBCs are non-nucleated cells with the plasma membrane as the only structural component. WBCs are generally non-proliferative in normal condition and have small and tightly packed nuclei. Electrical pulses are expected to induce membrane poration in RBCs, WBCs, and CTCs. However, nucleus poration is responsible for the observed nucleus expansion of CTCs.

Nucleus poration is a mechanism for selective purging of CTCs. Using an electroporative microfluidic device (FIG. 1A), selective purging of CTCs is demonstrated. At a constant electric field intensity of 500 V/cm, 200 ms exposure duration, and a flow rate of 0.28 ml/min, up to 100% of CTC death is achieved with approximately 10% of WBC death and no detectable RBC death (FIG. 1F).

### Example 2: A Switched Polarity Transferring Pulse Reduces the Viability of CTCs but Increases the Viability of PBMCs

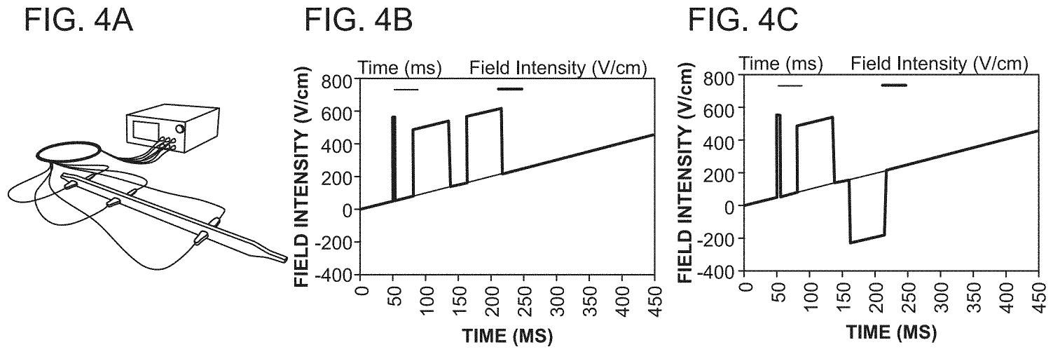

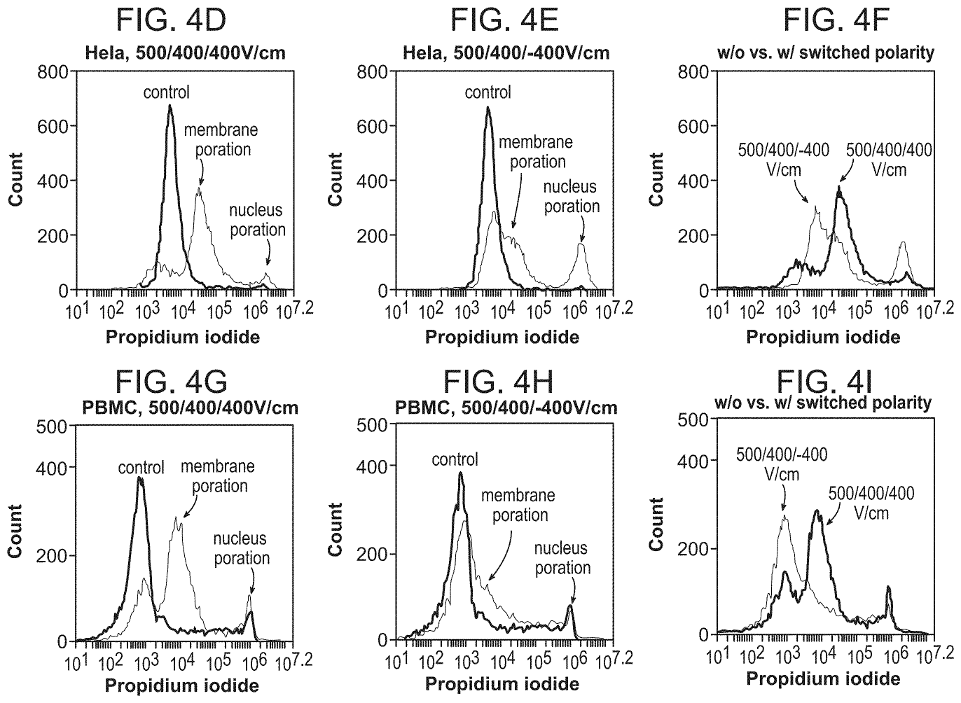

A flow electroporator device described herein is designed to purge CTCs in extracorporeal blood of metastatic cancer patients during hemodialysis (FIG. 4A). The polarity of the electrical pulses was modulated to enhance the lysis of CTCs while improving the viability of blood cells. In one example, cells traversing the flow electroporator device at 500 ml/min experienced three sequential electrical pulses: a poring pulse of 500 V/cm for 5 ms and two identical transferring pulses of 400 V/cm for 50 ms (FIG. 4B). A peristaltic pump was used to control the flow rate from 0.06 to 3400 mL/min, where a rotor compresses the flexible pump header tubing as it rotates. The rotational speed of the pump head determines the flow rate through the arterial bloodline tubing with an integrated flow electroporator device (FIG. 3A & 3B). In another example, cells traversing the flow electroporator device at 500 ml/min experienced three sequential electrical pulses: a poring pulse of 500 V/cm for 5 ms, a transferring pulse of 400 V/cm for 50 ms, and a switched polarity transferring pulse of −400 V/cm for 50 ms (FIG. 4C). Propidium iodide (PI) was used as DNA stain to evaluate cell viability using flow cytometry. Treatment of CTCs with the condition that does not include a switched polarity transferring pulse led to two distinctive CTC populations: a population with low PI staining due to membrane poration and a population with high PI staining due to nucleus poration (FIG. 4D). Interestingly, treatment of CTCs with the condition that included a switched polarity transferring pulse reduced the CTC population with membrane poration but increased the CTC population with nucleus poration (FIG. 4E & 4F). In contrast, treatment of PBMCs with the condition that did not include a switched polarity transferring pulse increased only the PBMC population with membrane poration (FIG. 4G). Treatment of PBMCs with the condition that included a switched polarity transferring pulse significantly reduced the PBMC population with membrane poration (FIG. 4H & 4I). Clearly, membrane poration is reversible, while nucleus poration is aggravated, with a switched polarity transferring pulse. The switched polarity transferring pulse reduced the viability of CTCs but increased the viability of PBMCs.

### Example 3: Electrolytes Increase Lysis of CTCs While Having No Effect on the Viability of PBMCs

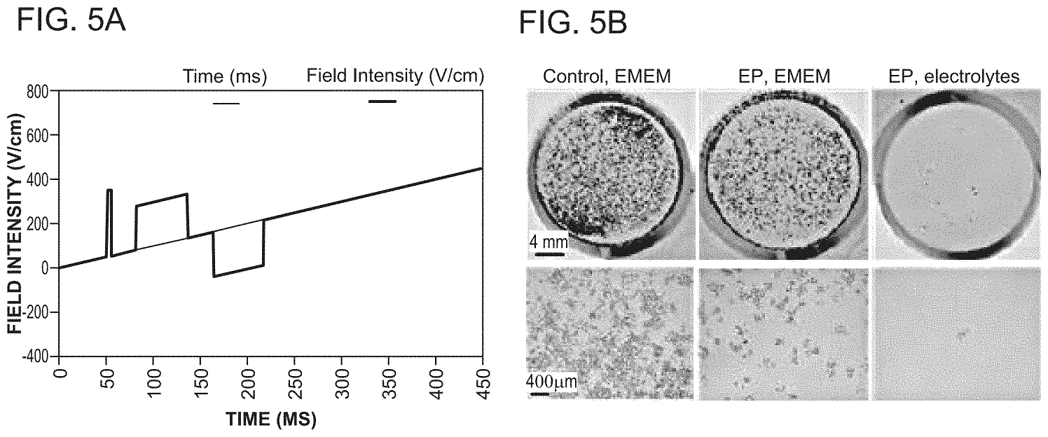

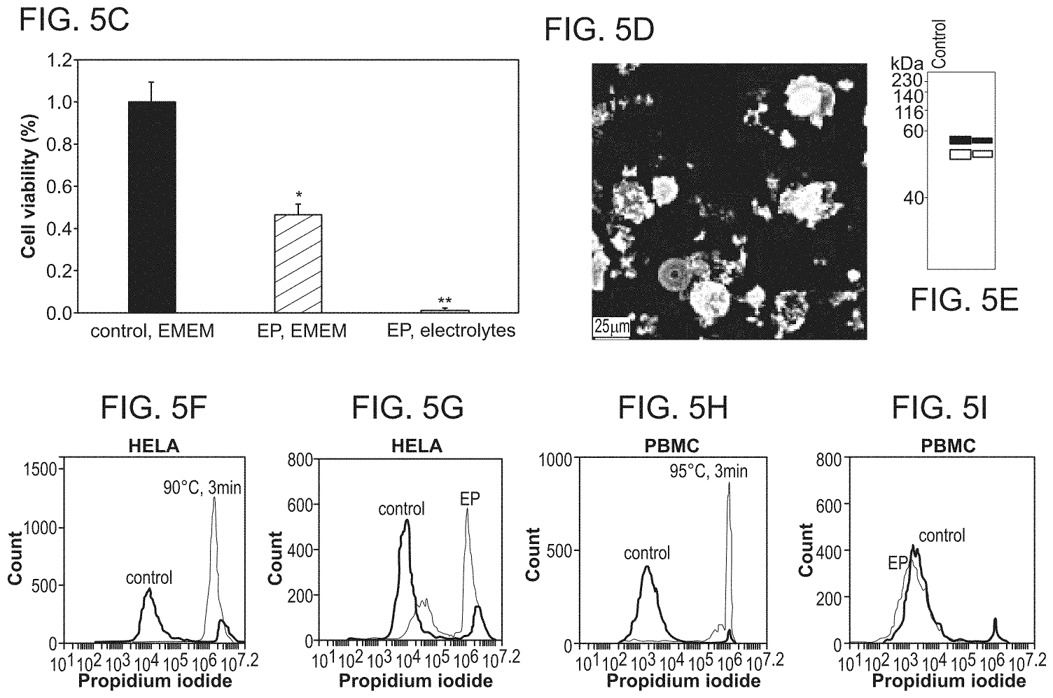

Selective purging of CTCs was further enhanced with electrolyte supplementation. To enhance the electric current of the flow electroporator device, CTCs were suspended in an electrolyte solution, which comprised Eagle's Minimum Essential Medium (EMEM) supplemented with NaCl, CaCl₂, and KCl with final concentrations of 270 mM, 70 mM, and 46 mM, respectively. CTCs were treated with the flow electroporator device using sequential electric field intensities of 300 V/cm for 5 ms, 200 V/cm for 50 ms, and −200 V/cm for 50 ms (FIG. 5A). Crystal violet staining was used to assay for cell viability (FIG. 5B). Without electrolyte supplementation, treatment of CTCs with the flow electroporator device reduced cell viability by approximately 60% compared to untreated control (FIG. 5C). With electrolyte supplementation, treatment of CTCs with the flow electroporator device reduced cell viability by more than 99.9% compared to untreated control. Lysis of CTCs in an electrolyte solution was confirmed with confocal fluorescence imaging (FIG. 5D) and Western blots of released housekeeping proteins HSP60 and β-actin (FIG. 5E). Flow cytometry assays using PI staining revealed that approximately 70% of CTCs experienced nucleus poration and the remaining 30% experienced membrane poration (FIG. 5F & 5G). In contrast, there was no observable effect on the viability of PBMCs in an electrolyte solution following the treatment with the flow electroporator device (FIG. 5H & 5I). Electrolyte supplementation enhanced lysis of CTCs by the flow electroporator device at low electric field intensities, while having no effect on the viability of PBMCs.

### Example 4: Drug Delivery via Electroporation

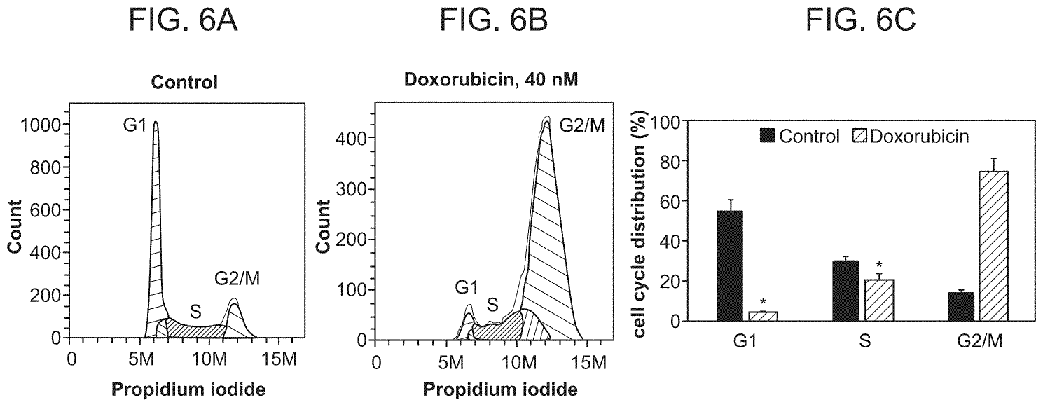

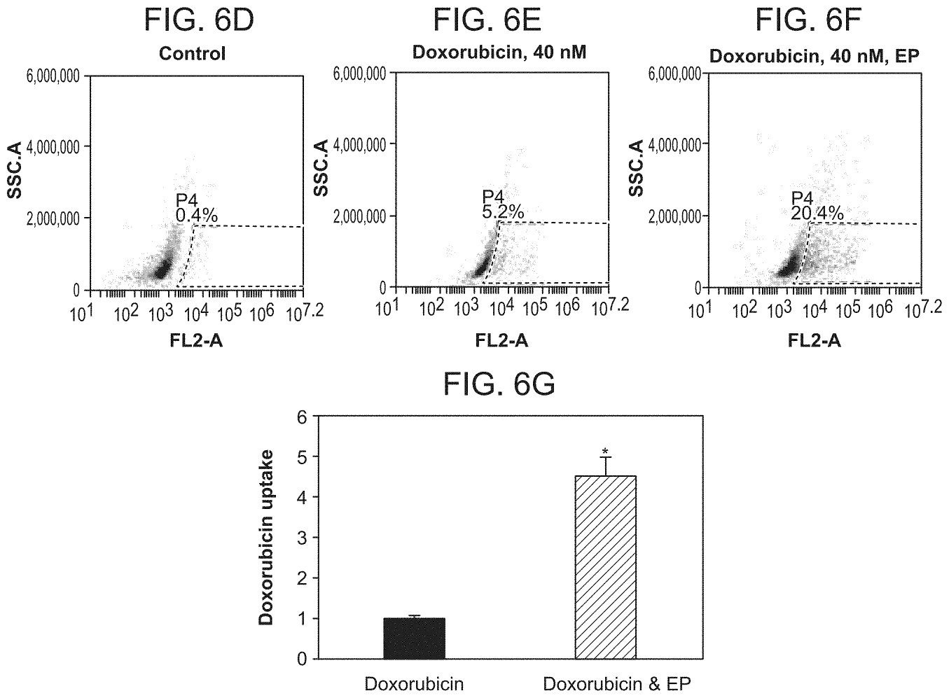

Another utility of the flow electroporator device is drug delivery during hemodialysis. Due to the unique cancer hallmarks of CTCs, many chemotherapeutic agents can be used for their selective targeting. For example, doxorubicin inhibited DNA replication and caused cell cycle arrest of CTCs, which have a high proliferation rate (FIG. 6A-C). Blood cells were less susceptible to the effects of doxorubicin because RBCs do not have DNA and WBCs are generally non-proliferative in normal condition. Electroporation by the flow electroporator device enhanced doxorubicin uptake by CTCs by more than 4 fold (FIG. 6D-G). An advantage of drug delivery during hemodialysis is the subsequent removal of excess chemotherapeutic agents, thereby minimizing unnecessary side effects to cancer patients.

### Example 5: DGAT1 Inhibitor Reduces the Viability of CTCs in Blood Plasma

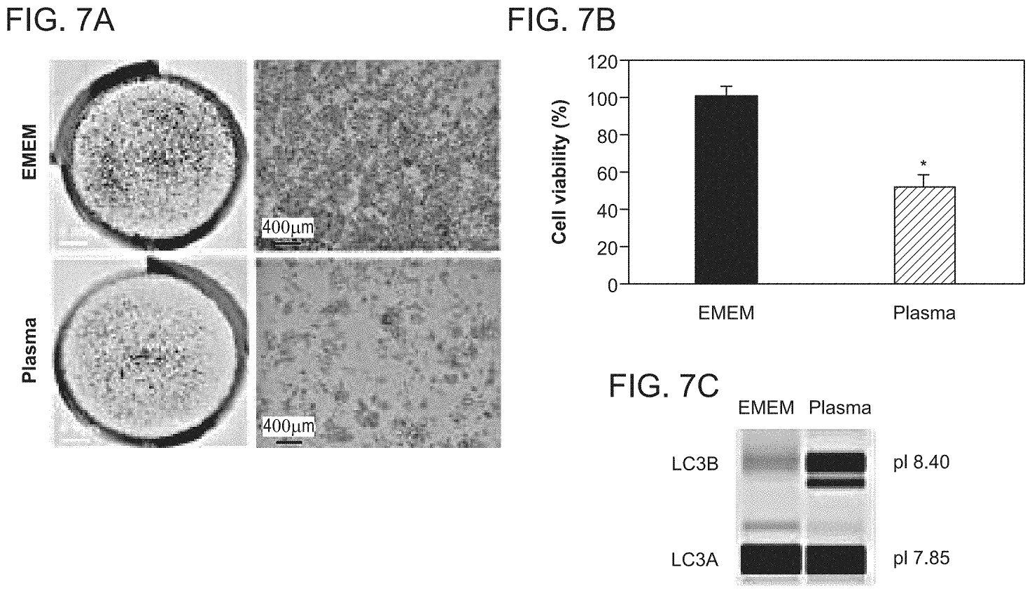

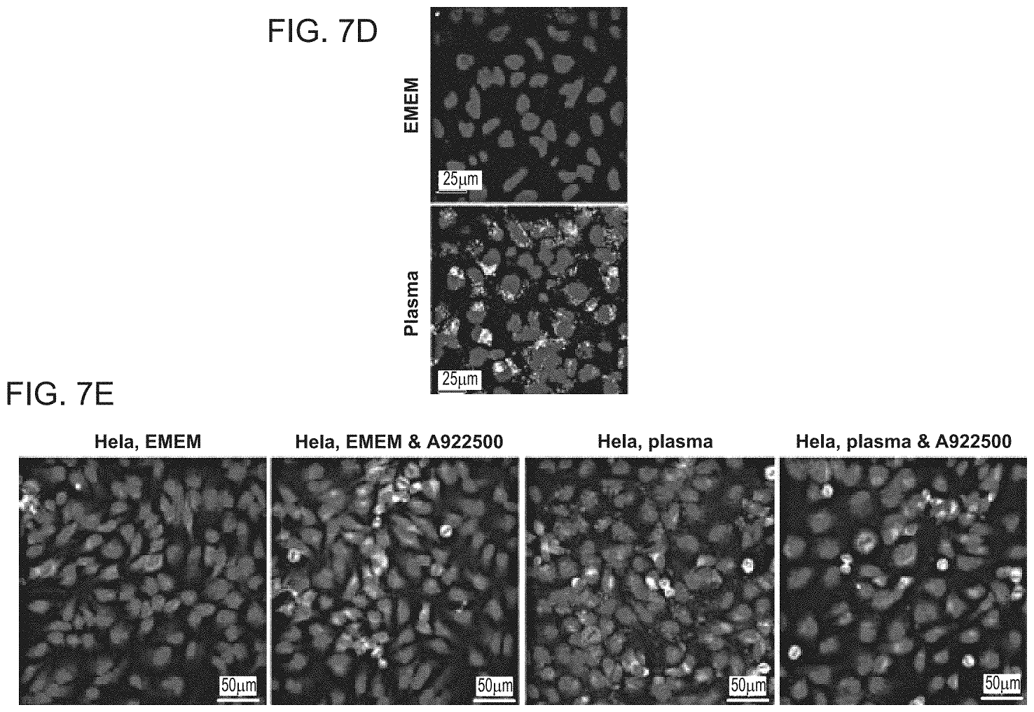

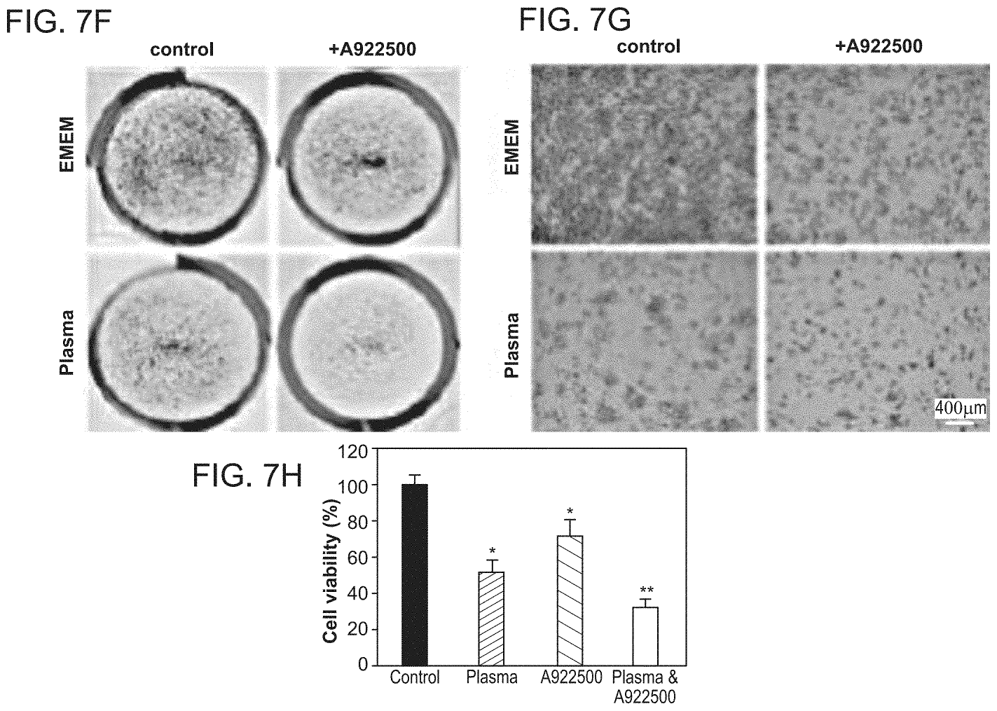

Using the flow electroporator device to deliver small molecule inhibitors of lipid droplet biosynthesis provides an additional mechanism for selective targeting of CTCs. The bloodstream is a highly toxic environment to CTCs due to the abundance of bioactive lipids and free fatty acids. Incubation of CTCs with blood plasma for 24 hours reduced the viability of CTCs by nearly 50% (FIG. 7A & 7B). Blood plasma induced autophagic cell death of CTCs as indicated by the increased expression of LC3B, an autophagosome biomarker (FIG. 7C), and the number of autophagosomes (FIG. 7D). As a defensive mechanism, CTCs converted bioactive lipids and free fatty acids into neutral triacylglycerols and stored them in cytoplasmic lipid droplets (FIG. 7E, third panel). DGAT1 is an enzyme that catalyzes the conversion of diacylglycerol and fatty acyl-CoA to triacylglycerol. A922500, an inhibitor of DGAT1, inhibited lipid droplet accumulation in CTCs incubated with blood plasma (FIG. 7F, fourth panel). Treatment with A922500 further reduced the viability of CTCs in blood plasma. In contrast, blood cells did not have the capability to store cytoplasmic lipid droplets and were generally unaffected by A922500.

### Example 6: Gene Delivery via Electroporation

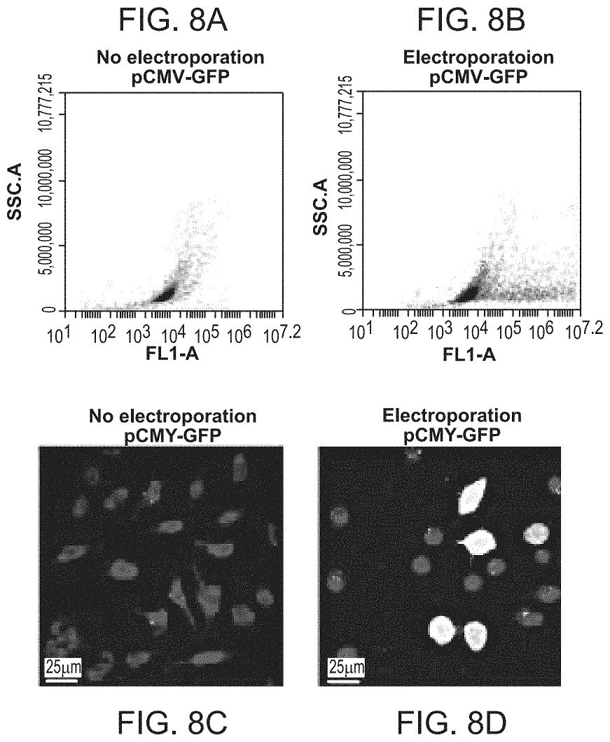

The utility of the flow electroporator device for gene delivery was demonstrated. A pCMV-GFP plasmid was successfully delivered to CTCs suspended in EMEM medium using the flow electroporator device, which applied sequential electric fields of 300 V/cm for 5 ms, 200 V/cm for 50 ms, and −200 V/cm for 50 ms at a flow rate of 500 ml/min. Transfection efficiency, as determined by the percentage of CTCs expressing green fluorescent protein, was approximately 25% (FIG. 8A-D). The flow electroporator device allowed efficient delivery of nucleic acid-based therapeutics to CTCs and blood cells in extracorporeal blood of cancer patients during hemodialysis.

It is thus apparent that there is provided in accordance with the present disclosure, systems, methods, and devices for therapeutic targeting of circulating tumor cells during hemodialysis. Many alternatives, modifications, and variations are enabled by the present disclosure. While specific embodiments have been shown and described in detail to illustrate the application of the principles of the present invention, it will be understood that the invention may be embodied otherwise without departing from such principles. Accordingly, Applicant intends to embrace all such alternatives, modifications, equivalents, and variations that are within the spirit and scope of the present invention.

All publications and patents cited in this disclosure are incorporated by reference in their entirety. To the extent, the material incorporated by reference contradicts or is inconsistent with this specification, the specification will supersede any such material. The citation of any references herein is not an admission that such references are prior art to the present disclosure. Various terms relating to aspects of the description are used throughout the specification and claims. Such terms are to be given their ordinary meaning in the art unless otherwise indicated. Other specifically defined terms are to be construed in a manner consistent with the definitions provided herein.

The articles “a” and “an” are used herein to refer to one or more than one (e.g., to at least one) of the grammatical object of the article. By way of example, “an element” means one element or more than one element.

The term “about” when referring to a measurable value such as an amount, a temporal duration, and the like, is meant to encompass variations of ±20% or less, or in some instances ±15% or less, or in some instances ±10% or less, or in some instances ±5% or less, or in some instances ±1% or less, or in some instances ±0.1% or less, from the specified value, as such variations are appropriate.

The phrase “and/or” as used herein should be understood to mean “either or both” of the elements so conjoined, e.g., elements that are conjunctively present in some cases and disjunctively present in other cases. Multiple elements listed with “and/or” should be construed in the same fashion, e.g., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

1. A method for therapeutically targeting circulating tumor cells during hemodialysis, the method comprising: connecting a patient's bloodstream to a flow electroporator device, the device comprising:
a. an array of polymeric channels, wherein the opposing sidewalls of each channel are lined with discontinuous sections of electrodes, interspersed by non-conductive polymer sections to generate sequential electric fields by constant direct current voltages;
b. sequential electric fields with independently regulated intensity, duration, and polarity to induce lysis of circulating tumor cells and deliver chemotherapeutic agents to reduce the viability of circulating tumor cells; and
c. polymeric tubing inlet and outlet for integration into the bloodline tubing of any hemodialysis machine

2. The method of claim 1, wherein the polymer is selected from the group consisting of acrylonitrile butadiene styrene, polyamide, polycarbonate, polyethylene, polymethyl methacrylate, polypropylene, and polyvinyl chloride.

3. The method of claim 1, wherein the electrode is selected from the group consisting of aluminum, chrome, cobalt, copper, gold, magnesium, nickel, palladium, platinum, silver, stainless steel, and titanium.

4. The method of claim 1, wherein the number of polymeric channels is between about 1 and about 1024.

5. The method of claim 1, wherein the height and/or width of each channel is between about 1 micrometer and about 100 millimeters.

6. The method of claim 1, wherein the blood flow rate through each channel is between about 0.06 ml/min and about 3400 ml/min.

7. The method of claim 1, wherein the length of the electrodes is between about 1 nanometer and about 1 meter.

8. The method of claim 1, wherein the number of sequential electric fields is between about 1 and about 10,000.

9. The method of claim 1, wherein the electric field intensity is between about 5 mV/cm and about 5 kV/cm.

10. The method of claim 1, wherein the electrical pulse duration is between about 1 picosecond and about 10 hours.

11. The method of claim 1, wherein circulating tumor cells are lysed by sequential electrical pulses.

12. The method of claim 1, wherein lysis of circulating tumor cells is enhanced with switched polarity electrical pulses.

13. The method of claim 1, wherein lysis of circulating tumor cells is enhanced with electrolytes.

14. The method of claim 13, wherein the electrolytes are selected from a group comprising sodium, calcium, potassium, chloride, phosphate, magnesium, or a combination thereof.

15. The method of claim 14, wherein molar concentrations of the electrolytes are between about 1 millimolar and about 1 molar.

16. The method of claim 1, wherein the chemotherapeutic agents are delivered into circulating tumor cells and blood cells using sequential electrical pulses.

17. The method of claim 16, wherein the chemotherapeutic agents are selected from the group consisting of electrolytes, small chemical molecules, proteins, RNAs, DNAs, and combinations thereof.

18. The method of claim 1, wherein lysis of the circulating tumor cells and delivery of chemotherapeutic agents to reduce the viability of circulating tumor cells using sequential electrical pulses are performed individually, sequentially, simultaneously, or in combination with other methods of purging circulating tumor cells from extracorporeal blood.

19. The method of claim 18, wherein debris of lysed circulating tumor cells and excess chemotherapeutic agents are removed from extracorporeal blood via dialysis.
